(19) United States
(12) Patent Application Publication
(10) Pub. No.: US 2004/0167426 A1
(43) Pub. Date: Aug. 26, 2004

(54) Method and device for locating visceral constrictions

(76) Inventor: Gaston Vantrappen, Overijse (BE)

Correspondence Address: KNOBBE MARTENS OLSON & BEAR LLP, 2040 MAIN STREET, FOURTEENTH FLOOR, IRVINE, CA, 92614, US

(21) Appl. No.: 10/728,074

(22) Filed: Dec. 4, 2003

(30) Foreign Application Priority Data
Dec. 5, 2002 (EP) 02447243.3

(51) Int. Cl. A61B 5/00

(52) U.S. Cl. 600/560

(57) ABSTRACT

A method and device are provided for determination of the location of an anatomical or functional constriction of a hollow body organ, such as the lower esophageal sphincter (LES) in the gastroesophageal junctional segment, or constrictions in other tubular organs of the body such as other parts of the gastrointestinal tract, the pancreatico-biliary tract, the genito-urinary tract, and blood vessels.

### FIELD OF THE INVENTION

A method and device are provided for determination of the location of an anatomical or functional constriction of a hollow body organ, such as the lower esophageal sphincter (LES) in the gastroesophageal junctional segment, or constrictions in other tubular organs of the body such as other parts of the gastrointestinal tract, the pancreatico-biliary tract, the genito-urinary tract, and blood vessels.

### BACKGROUND OF THE INVENTION

The lower esophageal sphincter (LES) has a crucial role in the prevention of gastroesophageal reflux (GER) and the pathological effects thereof. The term gastroesophageal reflux refers to the backflow of gastric contents, particularly of gastric acid and bile, from the stomach, into the esophagus. When this reflux occurs too often or lasts too long, it results in symptoms and lesions of gastroesophageal reflux disease (GERD).

The quantitative evaluation of gastroesophageal reflux is based on measurement of intraesophageal acidity and bile concentration, using devices (such as pH electrodes, acid exposure sensors, and bile sensors) that have to be positioned at a defined distance above the upper border of the LES. Therefore, the first step in the quantification of abnormal gastroesophageal reflux is the determination of the location of the upper border of the lower esophageal sphincter.

Determining the location of a constriction in a hollow organ is a problem which is applicable to other organs as well. In general, it is often necessary for the physician to identify and indicate the location of a visceral constriction by the number of centimeters an exploring device has to be introduced into the body in order to reach the site of the constriction in the visceral tube, because that is the spatial reference for the application of the diagnostic investigation or therapeutic procedure which is to be performed once the location has been determined.

The term ‘constriction’ as used herein is a broad term and is used in its ordinary sense, including, without limitation, a localized decrease of the lumen of a hollow organ or a localized decrease of distensibility of such an organ. Such a constriction can be anatomical or functional. An anatomical constriction is caused by a fibrotic, edematous, inflammatory, tumoral or plaque-forming process in the wall of the hollow organ or it can be due to normal structures or pathological processes in the tissues surrounding the said organ. A functional constriction is due to the contractile state of the muscles of the hollow organ, such as that which occurs in sphincteric segments.

In the specific case of the gastro-esophageal functional segment, several techniques have been proposed to locate the lower esophageal sphincter (LES). The radiological identification of the LES requires a fluoroscopic examination by a specialized radiologist and imposes ionizing radiation upon the patient. Moreover, the visualization and location of the LES on radiological images does not allow positioning of an exploring device at a defined level above the sphincter, unless the device is introduced and positioned in the esophagus during the radiological examination.

Endoscopy frequently does not permit determination of the upper limit of the lower esophageal sphincter. Moreover, endoscopy is an invasive and specialized examination.

The pH step-up technique, such as it is described by Mattox et al., Digestive Diseases and Sciences, Vol. 37, No. 8 (August 1992), pp. 1185-1191, has been shown to locate the proximal margin of the LES inaccurately.

The wall motion device was developed to detect those gastric antrum contractions that substantially narrow the lumen of the antrum without resulting in an occlusion of that lumen. The device is described in ‘Evaluation of patterns of human antral and pyloric motility with an antral wall motor detector’, American Journal of Physiology, 1990;258;G616-G623. The most important drawbacks of the wall motion device for determining constrictions in a hollow organ are, firstly, that it measures the diameter changes in a given plane of hollow organ, not the volume changes, and, therefore, will fail to detect a flattening of the lumen which leaves the diameter of that lumen unchanged in the plane of the detector. Secondly, its dimensions are such that they make the introduction through the nose very difficult and often impossible. Thirdly, the technique is too complicated to be used for clinical purposes.

It is believed that the most accurate, currently available way of locating the upper border of the lower esophageal sphincter is by manometric identification of the lower esophageal high pressure zone, which corresponds to the lower esophageal sphincter. Manometry, therefore, is at present the first step in the process of measuring gastroesophageal reflux. Manometry is a technically complex laboratory investigation which is only available in highly specialized centers and is expensive. Even if a pH electrode and a pressure measuring device (solid state or perfused catheter) are combined in a single probe, as described by S. Singh et al., American Journal of Gastroenterology, 1992, Vol. 87, pp. 967-970; Devault and Castell, American Journal of Gastroenterology, 1991, Vol. 86, pp. 380-381; Klingler et al., American Journal of Gastroenterology, 2000, Vol. 95, No. 4; and in document U.S. Pat. No. 5,117,827, the cost and complexity of this examination remains prohibitive. This is the reason why the use of manometric techniques (including the LES locating device described by Singh et al.) is limited to highly specialized centers. Esophageal pH measurement, therefore, is at present not available for a large number of patients with gastroesophageal reflux disease for whom this examination would be very useful.

### SUMMARY OF THE INVENTION

In general, treatment of constrictions in hollow viscera, due to processes such as fibrotic stricture, tumor growth or plaque formation, often requires local invasive procedures such as dilation, stenting or irradiation. Heretofore, accurate location of constrictions in other parts of the gastrointestinal tract, in the biliopancreatic or genitourinary tract, or in blood vessels has usually been based on techniques similar to the above-described techniques for determining the location of the lower esophageal sphincter, namely endoscopy, manometry and radiological, and MRI imaging.

Therefore, there is a need for a device that permits determination of visceral constrictions in a simple, non-expensive way, without manometry, so that it becomes accessible to all patients who need it.

The preferred embodiments provide a method and device which can determine the location of a visceral constriction in a hollow organ in a simple and inexpensive way, so that it can be used in a medical office outside a hospital environment, without the help of radiological, endoscopic or manometric techniques.

In a first embodiment, a device for determining a location of a visceral constriction in a body is provided, the device comprising a catheter comprising a compressible element having a volume dependent on compression of the element; a volume detector for detecting a change in volume of the compressible element; and a position detector for detecting a position of the compressible element with respect to a reference position measured along a length of the catheter.

In an aspect of the first embodiment, the compressible element comprises a balloon filled with a liquid or a gas.

In an aspect of the first embodiment, an interior of the balloon is in fluid communication with an interior of the catheter, and the device further comprises: a container having a variable volume; a measurement and read-out device for detecting a volume change in the container by measuring a physical value related to the volume change, and displaying or recording the physical value; a first tube connecting the catheter to the container; a second tube connecting the catheter to the measurement and read-out device; and a stopcock, wherein the stopcock can be placed in a first position or a second position, wherein when the stopcock is placed in the first position a connection between the balloon and the container is opened and a connection between the balloon and the measurement and read-out device is blocked, and wherein when the stopcock is placed in the second position a connection between the balloon and the measurement and read-out device is opened and a connection between the balloon and the container is blocked.

In an aspect of the first embodiment, the container comprises a vessel equipped with a piston.

In an aspect of the first embodiment, the container comprises a balloon.

In an aspect of the first embodiment, the compressible element comprises a compressible material which is electrically conductive, and wherein an electrical resistance of the compressible material is dependent on a compression of the compressible material.

In an aspect of the first embodiment, the device further comprises: a first electrode and a second electrode, wherein the first electrode and the second electrode are attached to the element; a first conductor in electrical connection to the first electrode; and a second conductor in electrical connection to the second electrode; wherein the measurement and read-out device is electrically connected to the first conductor and the second conductor is capable of measuring an electrical resistance between the first electrode and the second electrode, and is capable of displaying and/or recording the electrical resistance.

In an aspect of the first embodiment, the element comprises an electrically conductive foam enveloped by a membrane.

In an aspect of the first embodiment, the element comprises a capsule containing electrically conductive granules.

In an aspect of the first embodiment, the element comprises a closed balloon, wherein the closed balloon comprises a first chamber and second chamber connected by a tube, wherein at least one of the first chamber and the second chamber comprises a transducer which is capable of detecting a liquid being pressed from the first chamber into the second chamber.

In an aspect of the first embodiment, the device further comprises: a first electrode and a second electrode; a first conductor in electrical connection to the first electrode; and a second conductor in electrical connection to the second electrode; wherein the measurement and read-out device is electrically connected to the first conductor and the second conductor is capable of measuring an electrical resistance between the first electrode and the second electrode, and is capable of displaying and/or recording the electrical resistance, wherein the balloon is attached to the catheter along a length of the catheter, wherein the balloon is filled with an electrically conductive liquid, wherein the transducer is situated in the second chamber, wherein the transducer comprises a foam, wherein the transducer is equipped with the first electrode and the second electrode, such that in operation, when the catheter is inserted into the body, the first chamber is inserted firstly and the second chamber is inserted secondly.

In an aspect of the first embodiment, the element comprises a first and a second end, the element comprising a balloon situated at the first end and a rigid hollow body situated at the second end, wherein the balloon and the rigid body are connected by a tube, wherein a gas is contained within the element, wherein the rigid hollow body further comprises a compressible body which is separated from the gas by a membrane such that the compressible body is capable of being compressed by gas which is pressed out of the balloon and into the rigid hollow body, and wherein the compressible body comprises a compressible material, wherein the compressible material is electrically conductive, and wherein an electrical resistance of the compressible material is dependent on a compression of the compressible material.

In an aspect of the first embodiment, the compressible material is an electrically conductive foam.

In an aspect of the first embodiment, the compressible material comprises a plurality of electrically conductive granules.

In an aspect of the first embodiment, the device comprises: a first electrode and a second electrode, wherein the first electrode and the second electrode are situated on the compressible body; a first conductor in electrical connection to the first electrode; and a second conductor in electrical connection to the second electrode; wherein the measurement and read-out device is electrically connected to the first conductor and the second conductor is capable of measuring an electrical resistance between the first electrode and the second electrode, and is capable of displaying and/or recording the electrical resistance, such that in operation, when the catheter is inserted into the body, the balloon is inserted firstly, and the rigid hollow body is inserted secondly.

In a second embodiment, a method for determining the location of a visceral constriction is provided, the method comprising: inserting a device comprising a compressible element into a body until the compressible element has passed a visceral constriction; defining a reference point for a distance over which the device is inserted into the body; and pulling out the device while monitoring a volume change of the compressible element as a function of a distance the device has been pulled out of the body, wherein the distance the device has been pulled out of the body is measured with respect to the reference point, and wherein the volume change of the compressible element indicates when the compressible element has passed the visceral constriction, whereby the location of the visceral constriction is determined.

### BRIEF DESCRIPTION OF THE DRAWINGS

FIGS. 1a and 1b are schematic representations of a balloon-catheter-based device.

FIG. 2 is a schematic representation of a device based on a detector made of compressible foam.

FIG. 3 is a schematic representation of a device based on a detector made of a closed liquid-filled balloon.

FIGS. 4 and 5 are schematic representations of a device based on a gas-filled detector.

FIG. 6 shows results of an experiment, comparing a device of a preferred embodiment to a manometric device.

### DETAILED DESCRIPTION OF THE PREFERRED EMBODIMENT

The following description and examples illustrate a preferred embodiment of the present invention in detail. Those of skill in the art will recognize that there are numerous variations and modifications of this invention that are encompassed by its scope. Accordingly, the description of a preferred embodiment should not be deemed to limit the scope of the present invention.

The preferred embodiments are related to a device which comprises an element which has a variable volume, a means to pick up the volume changes that the element undergoes when it is pulled through a constriction in a body organ, and a measuring/displaying/recording system to show the volume changes which occur at each of the measuring levels during the pull-through procedure. A device of the preferred embodiments further comprises a means for measuring the distance over which the element has been pulled.

According to the preferred embodiments, the volume change detector can comprise a fluid- or gas filled balloon, a spongelike compressible body, or another structure which is able to undergo volume changes when it is pulled through a constriction in a hollow organ of the body and to re-assume its original volume when it is out of that constriction. The measuring system can be contained within the volume detector from where it is connected to an external (out of the body) displaying system. The measuring system can also be separate from the volume change detector but connected to it, inside or outside the body. The measuring system can also be connected to the displaying system, which shows the volume changes measured at various levels of the constriction during the pull-through procedure, thus allowing localization of the constriction in the hollow organ as the distance (for example, in centimeters) from the port of entry into the body onto the margins of the constriction.

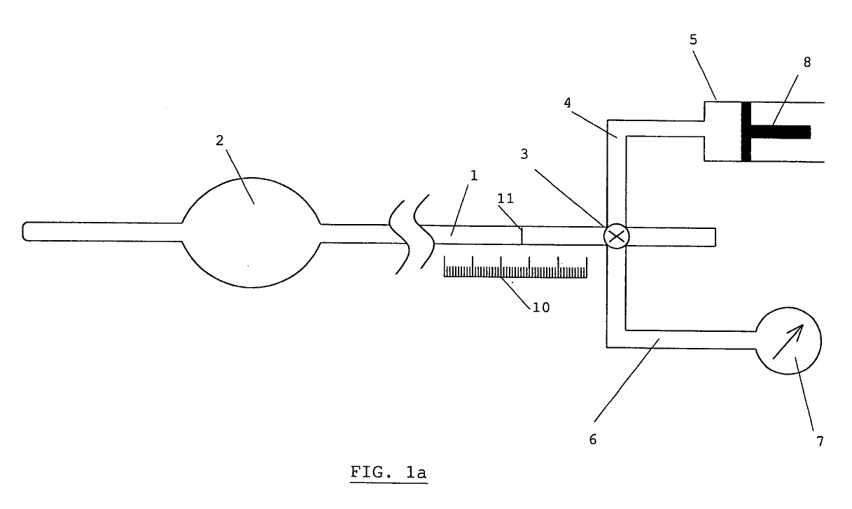

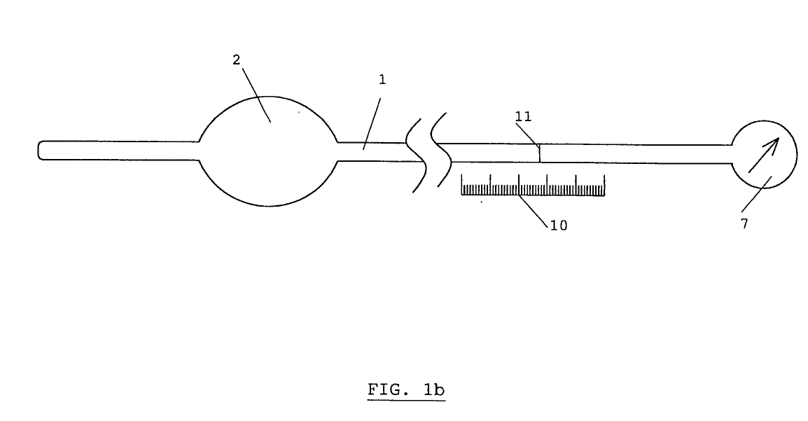

FIG. 1a shows a device according to a preferred embodiment. The device comprises a catheter 1, connected to a balloon 2, the interior of which is in connection with the interior of the catheter (namely, the balloon and catheter enclose a continuous hollow space). The catheter is equipped with a three-way stopcock 3, which is connected by a first connecting tube 4 to a container 5 with variable content, and by a second connecting tube 6 to a measurement and read-out device 7. A liquid is present in the device as shown in FIG. 1a. The container 5 can be a vessel equipped with a piston 8, which is the case in the FIG. 1a. The measurement and read-out device 7 can, for example, comprise a vertical liquid column, connected to the catheter and balloon, so that a volume change of the balloon can be translated into a change in liquid column height.

The stopcock 3 can be put in a first position, connecting the interior of the catheter 1 and balloon 2 with the interior of the container 5, while blocking the connection of the catheter 1 to the measurement and read-out device 7. In that way, the liquid can be induced to flow from the container to the balloon and vice versa. By moving the stopcock 3 to a second position, the interior of the catheter 1 and balloon 2 can be put in connection with the measurement and read-out device 7, while blocking the connection between the balloon 2 and the container 5.

The device operates in a straightforward way. Before insertion of the device, the liquid contained in the balloon 2 is induced to flow into the container 5, by the piston movement or by compression of balloon 2, thereby emptying the balloon 2. Deflating the balloon in this way facilitates insertion of the catheter and balloon into the body. For LES-location, the catheter is inserted through the nose of a patient, sufficiently far for the balloon to enter the stomach. At that point, the balloon is filled with liquid from the container 5 through the piston action, with the stopcock 3 in the first position. Then the stopcock 3 is moved to the second position, connecting the filled balloon with the measurement and read-out device 7. After that, the catheter and balloon are pulled out slowly. The device is equipped with a means for measuring the distance the balloon has been pulled out. This can be a simple ruler 10, along which the catheter is moved during the pull-through action, in combination with a marking 11 on the catheter, so that the distance over which the catheter has been pulled out can be recorded with respect to a reference position, for example the position wherein the marking 11 coincides with the beginning of the ruler 10. Of course, other and more sophisticated means for measuring the pull-out distance can be implemented as well, but it is imperative that this distance can be monitored. During the pull-through action, the balloon enters the LES at some point. The diameter of the liquid-filled balloon is larger than the diameter of the LES, so that the balloon is compressed when it is pulled through the closed LES, leading to a change in its volume. Liquid is pressed out of the balloon as a consequence, and flows towards the read-out device 7. Compression of the balloon is thereby detected, for example through a rise in height of a liquid column, at which point it is established that the balloon has entered the LES. When the pull-through procedure is continued, the balloon undergoes further compression, as it passes the LES. Leaving the LES, the balloon reassumes its former volume, which is detected as a decrease in liquid column height. The device of the preferred embodiments thus permits detection of the position of the LES, by recording the volume changes indicated on the read-out device, as a function of the pull-through distance. More specifically, the device allows to locate the upper border of the LES as a sharp decrease in the height of a liquid column, caused by the sudden return of the volume detector, namely, the balloon, to the volume it had before entering the constriction formed by the LES.

The device can be used to detect any other visceral constriction. The dimensions of the device, including length and diameter of catheter, dimensions of the balloon, and the like, can be adapted to every specific case.

The balloon is preferably made of a thin membrane. The balloon typically has a length of a few millimeters or less to a few centimeters or more, depending upon the nature and location of the constriction. The maximum diameter of the balloon is typically from about 3, 4, or 5 millimeters to about 6, 7, 8, 9, or 10 millimeters.

Instead of a piston-equipped vessel, the container 5 can be a second balloon. The container's volume can be increased or decreased according to the procedure which is performed. The container is able to accommodate the liquid pressed out of the balloon which is being pulled through a constriction. The volume of the external container 5 is preferably such that it easily receives the maximum volume of liquid contained in the detecting balloon 2.

In a simplified embodiment, the container 5 can be omitted from the device. The balloon is then inserted up to and through the constriction portion while the liquid is inside the balloon. In the case of the measurement device being based on a liquid column, this is generally possible. In this simplified embodiment (FIG. 1b), the stopcock can be omitted, a direct connection between the balloon 2 and the measurement and read-out device 7 is provided.

The catheter 1 is preferably made of a material which has a low compliance and is sufficiently flexible to be easily introduced in the hollow organ, but, at the same time, is sufficiently stiff to be pushed up or down the hollow organ. The catheter can be stiffened by any appropriate means, for example, a guiding wire (not shown in the Figures), so that it can be easily pushed up into the hollow organ of the body that is to be examined. The length of the catheter is adapted to the distance of the constriction in the hollow organ from the port of entry into the body and can vary from a few centimeters or less to one meter or more. The external diameter of the catheter is adapted to the nature of the organ which is to be explored but usually varies from 2 millimeters or less and 3 to 5 millimeters or more.

The measurement and read-out device 7 can work according to the principle of a liquid column, such as described above. However, any other device capable of measuring, directly or indirectly, the volume changes the detecting balloon undergoes when it is pulled through the constriction, can be implemented, in combination with a means for displaying and/or recording the measured value during the pull-through procedure. Generally, in a device of the preferred embodiments, these volume changes can be transduced into changes in electrical, optical or other physical variables which are a measure of said volume changes and which can be transmitted to an external read-out or recording device.

The device shown in FIGS. 1a and 1b is filled with an amount of liquid which is sufficient to allow the measuring device 7 to detect and measure the volume changes which occur in the internal balloon (for example, when it is pulled through a constriction in the hollow organ which is being examined). This amount of liquid depends upon the size of the internal balloon 2 and upon the length and diameter of the catheter 1.

Instead of filling the detecting balloon of FIGS. 1a and 1b with a liquid, the balloon can be filled with gas and connected via a catheter to an external measurement and read-out device. In the latter case, the measuring and read-out device 7 outside the body can consist of a device which is capable of measuring the volume of gas pushed out of the detecting balloon when this balloon passes a constriction. As an example, the measuring device can consist of an electrically conductive foam, which, when compressed by gas coming out of the detecting balloon, undergoes changes in electrical resistance that are determined by the degree of compression.

According to another embodiment, the detector does not consist of a fluid-filled balloon but of another type of compressible body, the volume changes of which are transduced into changes in electrical, optical or other physical variables which can be transmitted to an external read-out device.

In one preferred embodiment, the compressible body is made of a piece of an electrically conductive foam or sponge or a capsule containing electrically conductive granules. Any compressible body used in this preferred embodiment is characterized by high compressibility and low compression set, the latter meaning that the compressible body returns to its original volume after compression. Suitable elastomeric materials include, for example, conductive polyurethane or silicone. The materials preferably have an electrical resistance which is dependent on the degree of compression. Conductive foam products exist, for example, foams whose electrical resistance decreases with compression.

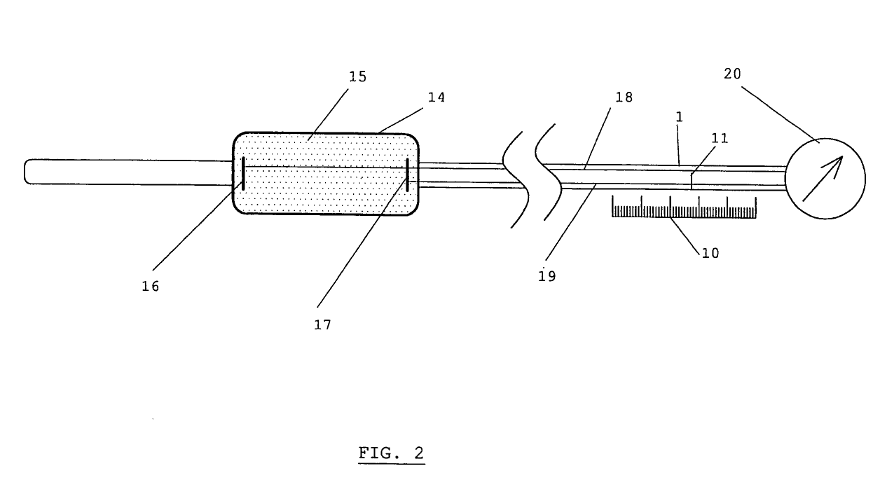

FIG. 2 shows a schematic view of a device according to this embodiment. At the end of a catheter 1, a cylinder-shaped piece of conductive foam 15, the detector, is attached to the catheter or incorporated into said catheter. The piece of foam 15 is preferably encapsulated by a membrane 14, to avoid the foam absorbing fluids. Two electrodes 16 and 17 are attached to the detector 15, at the extremities of this cylinder-shaped detector. Conducting wires 18 and 19 are present inside the catheter, connecting the electrodes 16 and 17 to an external measurement and read-out device 20. The latter device comprises at least a means for measuring the resistance between the electrodes 16 and 17. Any known device for measuring such a resistance value can be implemented, as well as a suitable device for displaying the measured value. As in the previous embodiment, a ruler 10 and marking 11 or any equivalent device is present, to detect the distance over which the catheter has been pulled out.

The procedure is also similar, namely, insertion of the device, for example through the nose, until the detector 15 has passed the visceral constriction, for example the LES. Then, a slow pull-through procedure is conducted wherein the electrical resistance value is recorded, for example, at every centimeter. Barring any significant compression of the detector, the resistance value remains virtually constant. When the detector 15 enters the constriction portion, a change in electrical resistance is recorded. After the constriction portion, the resistance returns to its original value. The resistance change allows location of the position of the visceral constriction.

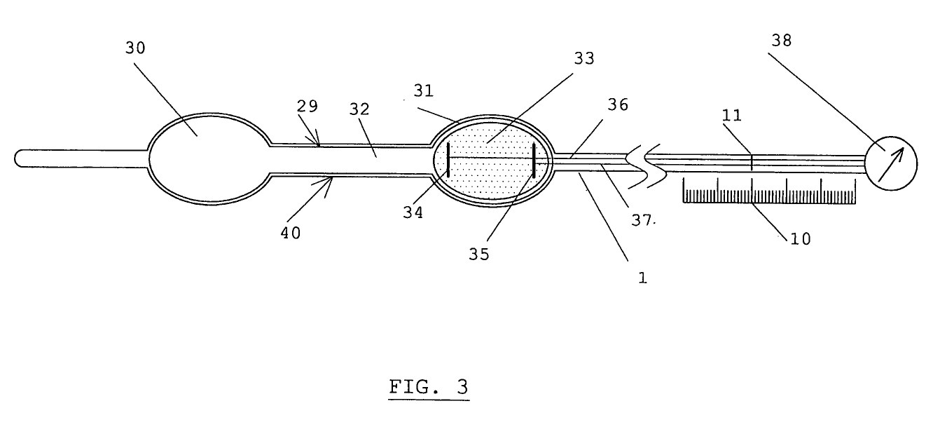

According to another embodiment, shown in FIG. 3, the device comprises a detector in the form of a closed balloon 29 consisting of two chambers 30 and 31, connected by a channel 32. The balloon is at least partially filled with a liquid and inserted in or otherwise connected to a catheter 1. When the device enters the body of a patient, the first chamber 30, which we call the distal chamber, enters first, followed by the second, ‘proximal’ chamber 31. The proximal chamber comprises a transducer which is able to detect the liquid that is being pressed from the distal chamber into the proximal chamber, when the distal chamber passes a constriction. In one preferred embodiment, the balloon is filled with an electrically conductive liquid, while the proximal chamber comprises a transducer in the form of a piece of foam 33, which practically fills that chamber 31. The foam is preferably of high compressibility and low compression set, and is equipped with two electrodes 34 and 35, connected by wires 36 and 37 to an external measurement and read-out device 38. The piece of foam 33 in this embodiment is possibly but not necessarily electrically conductive. When the liquid is pushed into the proximal chamber, the foam 33 is impregnated by the liquid, which influences the electrical resistance of the piece of foam 33. This change is picked up by the measurement device and displayed and/or recorded. The balloon is attached to a catheter 1, for example, by incorporating the balloon into a distal portion 40 of said catheter, said portion having the same or similar shape as the balloon.

The localization procedure takes place in the same way as described above. The catheter is inserted until both the distal and the proximal chambers of the balloon 29 have passed the constriction to be detected. In this way, the distal chamber 30 is emptied when it is pushed through the constriction, and subsequently filled when the proximal chamber 31 passes through the constriction. Then a pull-through procedure commences, in conjunction with a means 10, 11 for measuring the pull-out distance. The proximal chamber 31 is once more compressed during the pull-through, making sure thereby that the distal chamber 30 is sufficiently filled with liquid before said distal chamber is pulled through the constriction. This pulling through of the distal chamber 30 then allows a change in resistance to be recorded, which can then be translated into the location of the visceral constriction.

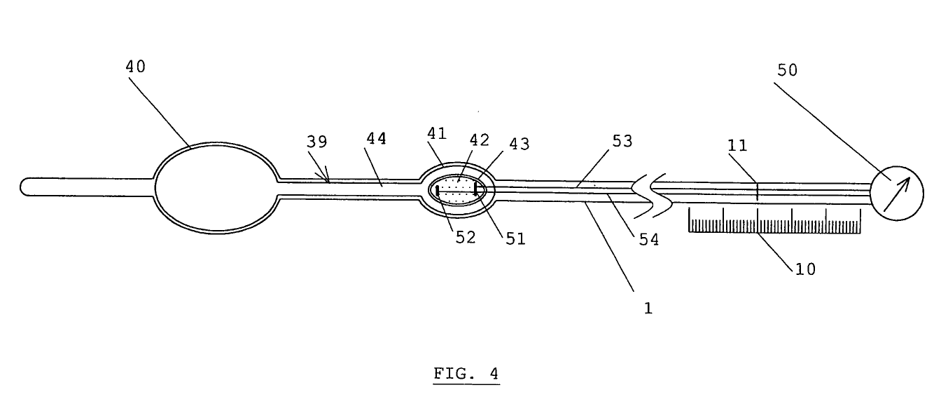

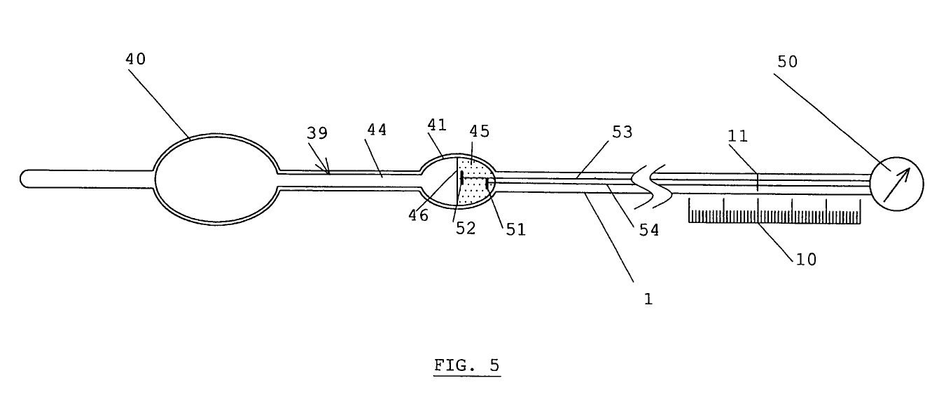

Instead of using a liquid-filled detector 29, a gas-filled detector 39 can be used (see FIG. 4). In this embodiment, the distal chamber is a compressible balloon 40, while the proximal chamber is a rigid capsule 41, containing a body which can be compressed by gas, such as a piece of electrically conductive foam 42 enveloped by a thin membrane 43. It is preferred that the proximal chamber 41 is sufficiently small in diameter so that it can be pulled through the constriction. The distal and proximal chamber are connected by a tube 44 to form one hollow body 39. In a slightly alternative form shown in FIG. 5, a piece of foam 45 can be used which is filling the proximal part of the capsule 41 but which is separated from the gas coming from the compressed distal chamber 40 by a thin membrane 46. Preferably, the compressible body (42 or 45) in the proximal chamber is preferably of high compressibility and low compression set. The compressible body (42 or 45) has an electrical resistance that is dependent on the degree of compression, which in turn depends on the volume of gas compressing it. The piece of foam is connected to the measurement and read-out system 50 through electrodes (51, 52) and conductors (53, 54), in an analogue way as in previous embodiments (FIGS. 2 and 3). The localization procedure is the same as the one described for the device of FIG. 3.

In the embodiments of FIGS. 2 to 5, the catheter has the same characteristics than in the balloon-equipped device, and can be produced from the same materials.

The device of the preferred embodiments can be modified as appropriate. For example, the distance between electrodes 16, 17 or 34, 35 or 51, 52 can vary. More than two electrodes might be present, and/or the electrodes might be placed circumferentially around the foam element 15 Instead of placed at either end of the element. Also, this element is not limited to a cylinder-like shape.

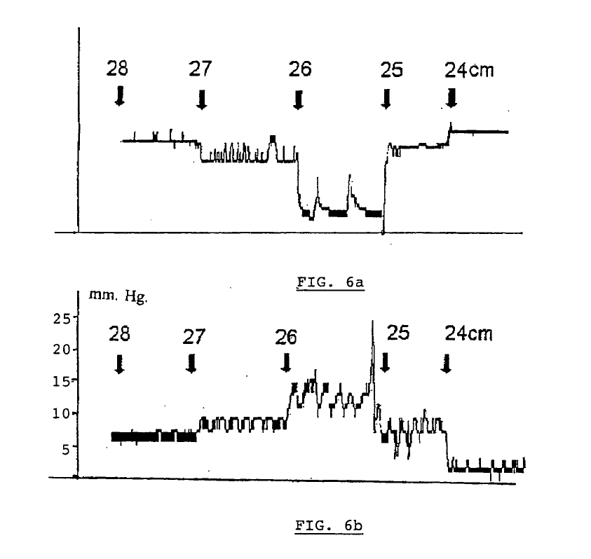

As an illustration of the performance of a device of the preferred embodiments, reference is made to FIGS. 6a and 6b. The graphs show the results of an experiment performed on an anesthetised cat. The lower graph (FIG. 6b) shows a manometric recording of the lower esophegeal high pressure zone. The pressures were measured using a stepwise pull-through technique and a Dent sleeve (Dent, J., Am. J. Gastroenterology, 1976, Vol. 71, pp. 263-267). The distances indicated in cm describe the distance d between the cat's incisors and the manometric device. The LES is located between 27 and 24 cm from the incisors.

The upper graph (FIG. 6a) shows a volumetric recording performed with a balloon-equipped device of the preferred embodiments such as the one shown in FIG. 1a. The measurement was performed with an endoluminal electrical impedance catheter with ring electrodes spaced at intervals of 1 cm (Silny et al, ‘Verification of the intraluminal multiple electrical impedance measurement for the recording of gastro-intestinal motility’, Journal of Gastro-Intestinal Motility, 1993, 5, pp. 107-122. This device was used in order to translate the height of the fluid column into the read-out signal which is shown in the graph. The recording represents, in a qualitative way, the volume changes the balloon undergoes when it is pulled from the stomach, at 28 cm from the cat's incisors, through the lower esophageal sphincter into the esophagus, at 24 cm from the incisors. The zone of decreased volume extends from 27 cm to 24 cm from the incisors and corresponds well with the ‘high pressure zone’, manometrically determined in the same cat during the same experiment (FIG. 6b).

It can be concluded from the experiment illustrated in FIG. 6 that the LES is located with the same accuracy, using the device of the preferred embodiments, than it is using a manometric device, in particular a Dent sleeve.

The above description discloses several methods and materials of the present invention. This invention is susceptible to modifications in the methods and materials, as well as alterations in the fabrication methods and equipment. Such modifications will become apparent to those skilled in the art from a consideration of this disclosure or practice of the invention disclosed herein. Consequently, it is not intended that this invention be limited to the specific embodiments disclosed herein, but that it cover all modifications and alternatives coming within the true scope and spirit of the invention as embodied in the attached claims. All patents, applications, and other references cited herein are hereby incorporated by reference in their entirety.

What is claimed is:

1. A device for determining a location of a visceral constriction in a body, the device comprising:
a catheter comprising a compressible element having a volume dependent on compression of the element;
a volume detector for detecting a change in volume of the compressible element; and
a position detector for detecting a position of the compressible element with respect to a reference position measured along a length of the catheter.

2. The device according to claim 1, wherein the compressible element comprises a balloon filled with a liquid or a gas.

3. The device according to claim 2, wherein an interior of the balloon is in fluid communication with an interior of the catheter, and wherein the device further comprises:
a container having a variable volume;
a measurement and read-out device for detecting a volume change in the container by measuring a physical value related to the volume change, and displaying or recording the physical value;
a first tube connecting the catheter to the container;
a second tube connecting the catheter to the measurement and read-out device; and
a stopcock, wherein the stopcock can be placed in a first position or a second position, wherein when the stopcock is placed in the first position a connection between the balloon and the container is opened and a connection between the balloon and the measurement and read-out device is blocked, and wherein when the stopcock is placed in the second position a connection between the balloon and the measurement and read-out device is opened and a connection between the balloon and the container is blocked.

4. The device according to claim 3, wherein the container comprises a vessel equipped with a piston.

5. The device according to claim 3, wherein the container comprises a balloon.

6. The device according to claim 1, wherein the compressible element comprises a compressible material which is electrically conductive, and wherein an electrical resistance of the compressible material is dependent on a compression of the compressible material.

7. The device according to claim 6, wherein the device further comprises:
a first electrode and a second electrode, wherein the first electrode and the second electrode are attached to the element;
a first conductor in electrical connection to the first electrode; and
a second conductor in electrical connection to the second electrode;
wherein the measurement and read-out device is electrically connected to the first conductor and the second conductor is capable of measuring an electrical resistance between the first electrode and the second electrode, and is capable of displaying and/or recording the electrical resistance.

8. The device according to claim 6, wherein the element comprises an electrically conductive foam enveloped by a membrane.

9. The device according to claim 6, wherein the element comprises a capsule containing electrically conductive granules.

10. The device according to claim 1, wherein the element comprises a closed balloon, wherein the closed balloon comprises a first chamber and second chamber connected by a tube, wherein at least one of the first chamber and the second chamber comprises a transducer which is capable of detecting a liquid being pressed from the first chamber into the second chamber.

11. The device according to claim 10, the device further comprising:
a first electrode and a second electrode;
a first conductor in electrical connection to the first electrode; and
a second conductor in electrical connection to the second electrode;
wherein the measurement and read-out device is electrically connected to the first conductor and the second conductor is capable of measuring an electrical resistance between the first electrode and the second electrode, and is capable of displaying and/or recording the electrical resistance, wherein the balloon is attached to the catheter along a length of the catheter, wherein the balloon is filled with an electrically conductive liquid, wherein the transducer is situated in the second chamber, wherein the transducer comprises a foam, wherein the transducer is equipped with the first electrode and the second electrode, such that in operation, when the catheter is inserted into the body, the first chamber is inserted firstly and the second chamber is inserted secondly.

12. The device according to claim 1, wherein the element comprises a first and a second end, the element comprising a balloon situated at the first end and a rigid hollow body situated at the second end, wherein the balloon and the rigid body are connected by a tube, wherein a gas is contained within the element, wherein the rigid hollow body further comprises a compressible body which is separated from the gas by a membrane such that the compressible body is capable of being compressed by gas which is pressed out of the balloon and into the rigid hollow body, and wherein the compressible body comprises a compressible material, wherein the compressible material is electrically conductive, and wherein an electrical resistance of the compressible material is dependent on a compression of the compressible material.

13. The device according to claim 12, wherein the compressible material is an electrically conductive foam.

14. The device according to claim 12, wherein the compressible material comprises a plurality of electrically conductive granules.

15. The device according to any one of claims 12, wherein the device comprises:
a first electrode and a second electrode, wherein the first electrode and the second electrode are situated on the compressible body;
a first conductor in electrical connection to the first electrode; and
a second conductor in electrical connection to the second electrode;
wherein the measurement and read-out device is electrically connected to the first conductor and the second conductor is capable of measuring an electrical resistance between the first electrode and the second electrode, and is capable of displaying and/or recording the electrical resistance, such that in operation, when the catheter is inserted into the body, the balloon is inserted firstly, and the rigid hollow body is inserted secondly.

16. A method for determining the location of a visceral constriction, the method comprising:
inserting a device comprising a compressible element into a body until the compressible element has passed a visceral constriction;
defining a reference point for a distance over which the device is inserted into the body; and
pulling out the device while monitoring a volume change of the compressible element as a function of a distance the device has been pulled out of the body, wherein the distance the device has been pulled out of the body is measured with respect to the reference point, and wherein the volume change of the compressible element indicates when the compressible element has passed the visceral constriction, whereby the location of the visceral constriction is determined.
